(19) United States
(12) Patent Application Publication
(10) Pub. No.: US 2021/0196669 A1
(43) Pub. Date: Jul. 1, 2021

(54) CANNABIS-BASED COMPOSITIONS FOR THE TREATMENT OF AUTISTIC SPECTRUM DISORDERS

(71) Applicant: TO PHARMACEUTICALS LLC, Wilmington, County of New Castle, DE (US)

(72) Inventors: Lihi BAR-LEV SCHLEIDER, Moshav Yevul (IL); Sid TAUBENFELD, New York, NY (US)

(73) Assignee: TO PHARMACEUTICALS LLC, Wilmington, County of New Castle, DE (US)

(21) Appl. No.: 17/058,065

(22) PCT Filed: May 23, 2019
(86) PCT No.: PCT/IL2019/050584

§ 371 Date: Nov. 23, 2020

Related U.S. Application Data
(60) Provisional application No. 62/676,093, filed on May 24, 2018.

(51) Int. Cl. A61K 31/352; A61K 31/05; A61K 9/00; A61K 9/70; A61P 25/00; A61P 25/22

(52) U.S. Cl. CPC A61K 31/352; A61K 31/05; A61P 25/22; A61K 9/7023; A61P 25/00; A61K 9/006

(57) ABSTRACT

Provided are therapeutic products and methods applicable to the treatment of patients with autistic spectrum disorder (ASD) or partial symptoms of ASD, said the products and methods using certain cannabis-based compositions enriched in CBD with a minimum content of THC under specific regimens and types of administration.

### TECHNOLOGICAL FIELD

The present invention generally relates to therapeutic products and methods applicable to the treatment of autistic spectrum disorders (ASD) or partial symptoms of ASD.

### BACKGROUND

Medicinal value of cannabis is well documented in the professional literature. Cannabinoids, the active ingredients of cannabis, are present in significantly higher concentrations in resin-producing pistillate inflorescences of cannabis plants. Various types of cannabis, such as C. Sativa, C. Indica and C. Ruderalis, may contain more than 100 different types of cannabinoids in distinct concentrations and proportions. The two predominant types, the tetrahydrocannabinol (THC) and cannabidiol (CBD), have been related to a number of clinically beneficial effects attributed to analgesic, antiemetic, antioxidative, neuroprotective and anti-inflammatory activities thereof in mammalian and human cells and organisms.

The mammalian endocannabinoid system is signal transduction system predominantly acting in the brain, and also in the peripheral tissues. Several cannabinoid receptors have been identified so far, the most prominent being the cannabinoid receptors types 1 and 2 (CB₁ and CB₂). The endocannabinoid system has been implicated in maintenance of homeostasis of the normal mammalian physiology, including systems of movement control, pain, appetite, memory, immunity and inflammation, among others. This can explain the high therapeutic potential of exogenous cannabinoids and cannabis-based medicines and their broad clinical applications. Nonetheless, a rationalized use of exogenous cannabinoids still imposes significant challenges, among others, due to legal issues.

A number of oral formulations of cannabinoids are commercially available today by prescription for specific clinical indications. Marinol capsules containing dronabinol, a synthetic Δ⁹-THC isoform, in sesame oil have been approved in a number of countries as an antiemetic in cancer patients subjected to chemotherapy and patients with AIDS. Cesamet capsules with nabilone, a synthetic THC analog, have been approved as a Marinol substitute. More recent formulations Namisol tablets with pure THC and Arvisol tablets with pure CBD have been approved for Alzheimer's disease and chronic neural pain, and Sativex (nabiximols), an oral spray containing THC and CBD,—approved for multiple sclerosis.

The invention is relevant to a group of neurodevelopmental disorders collectively termed autistic spectrum disorders (ASD) characterized by a range of social communication impairments and repetitive stereotyped behaviors, often with onset during early childhood and persisting through life. According to the core Diagnostic and Statistical Manual of Mental Disorders (DSM) criteria the symptoms of ASD can be grouped into five main categories: (I) sensory behavior, (II) social relating. (III) body and object use, (IV) language and communication skills, and (V) social and adaptive skills.

Children with ASD exhibit a wide variety of additional clinical symptoms beyond the DSM definition criteria. For instance, studies have shown a higher prevalence of sleep disturbances. Many patients manifest disruptive behaviors that make interventions difficult and place considerable strain on families and caregivers.

At present, ASD has no cure. Existing treatments are based on extensive behavioral interventions combined with alternative therapies. In addition, certain comorbidities are treated with atypical antipsychotic medications and mood stabilizers. These, however, have low tolerability and questionable efficacy.

Cannabis is one promising candidate for treating or at least alleviating certain symptoms of ASD. THC enriched cannabis strains have been known to exert positive effects on social behavior, and also anxiety and pain. In the past few decades, knowledge has accumulated regarding the role of the endocannabinoid system and its signaling in many CNS disease conditions, thereby opening the route for therapeutic exploitation of endocannabinoid-oriented drugs for the treatment of mental disorders.

Specifically in ASD, studies in animal models suggested that the endocannabinoid system can be a relevant target for pharmacological interventions. Anecdotal reports suggested effects of various cannabinoid compounds in resistant behavioral problems. One example is a fast-track FDA approved CBD product—Epidiolex indicated for the treatment of Dravet syndrome, a rare and catastrophic treatment-resistant form of childhood epilepsy.

Along with the growing understanding of pathophysiological mechanisms of ASD, several assessment tools have been developed for this condition using the following parent or physician questionnaires: Aberrant Behavior Checklist-Community (ABC-C) questionnaire, Clinical Global Impressions-Improvement (CGI-I) used as screening tools for children suspected with ASD, Sensory Profile II (SP-2) questionnaire fir the assessment of sensory sensitivities, Children's Sleep Habit Questionnaire (CSHQ) for the assessment of sleep disturbances. Changes in the sleep architecture (e.g., amount of REM sleep) and brain activity during sleep of participating children are commonly assessed by an overnight EEG exam.

Overall, however, in terms of etiology and treatment ASD remains a challenging disease for patients and physicians. Lack of effective treatment for ASD urges the search for new therapies for this groups of disorders.

### GENERAL DESCRIPTION

The present invention addresses the above needs in providing a targeted therapeutic solution for ASD, including partial ASD symptoms, as well as an overall alleviation of burden of presentations and consequences of this disease. It has been presently demonstrated that certain type of cannabis-based compositions generally characterized as enriched in CBD can be effective for treating or alleviating ASD, and even more effective in terms of specific ASD symptoms (or partial symptoms) such as self-injury, rage attacks, sleep problems, anxiety and mood changes, social and reciprocity skills, hyperactivity and overall improvement of general quality of life of patients and parent (see Examples 2 and 3).

More specifically, effects of the CBD enriched compositions of the invention are presently exemplified in the form of oil extracts of certain cannabis strains, with proven clinical validity in retrospective and prospective studies in ASD patients. An exemplary member of this group is represented by the strain referred to herein as ‘Avidekel’ generally described in US Plant Patent Application No. 2014/259228 (US Continuation Application No. 2017/290286).

More specifically, Avidekel herein encompasses a group of variants descended from an original Avidekel strain or cultivar characterized by a particularly high content of CBD and low THC, starting from CBD:THC ratio of about 4:1, respectively, or more.

The presently used Avidekel strain comprises CBD:THC ratio of at least about 20:1.

In terms of CBD/THC content, a typical Avidekel strain can comprise as high as 15-20% CBD or more (w/w), and THC as 1-4% or less than 1% (w/w).

Avidekel strain can be further defined as a cannabis strain comprising CBD up to at least 15%, 16%, 17%, 18%, 19%, 20% or more, and THC up to 4%, 3%, 2%, 1%, 0.5% or less (w/w).

According to the invention, an extract of a phyto-derived material (flowers) of Avidekel can be provided in an oil form, wherein CBD/THC content is carefully monitored (also referred to herein as MCG preparation), and CBD is even more enriched up to extent of at least about 30%, and THC is about 1.5% (w/w) (see Example 1).

More precisely, such oil preparations of Avidekel can be defined as comprising CBD up to at least 20%, 21%, 22%, 23%, 24%, 25%, 26%, 27%, 28%, 29, 30% or more, and THC up to 2%, 1.9%, 1.8%, 1.7%, 1.6%. 1, 5%, 1.4%, 1.3%, 1.2%, 1.1%, 1% or less (w/w).

In terms of CBD/THC amount, a single drop of Avidekel oil of the invention characteristically comprises about 12 mg CBD and 0.6 mg THC, and 10 drops of Avidekel oil, being the maximal dose used in the present studies—about 240 mg CBD and 12 mg THC (see Table 1).

It should be appreciated that a cannabis-derived material can include additional types of cannabinoids, although at much lesser concentrations and proportions, which can potentially contribute to its clinical effects.

Safety and efficacy of the MCG preparations of the invention have been evaluated in two retrospective self-reported studies 53 and 190 patients with ASD (Example 2), and further in a double-blind, placebo controlled randomized clinical trial (Example 3). In these instances the MCG has been administered as a combination therapy, i.e., together with other concomitant drugs (herein also therapeutic agents).

Drug safety and efficacy have been presently evaluated using high quality assessment criteria, including: Clinical Global Impressions-Improvement (CGI-I) questionnaire, Sensory Profile II (SP-2) questionnaire Children's Sleep Habit Questionnaire (CSHQ), Aberrant Behavior Checklist-Community (ABC-C) questionnaire, Adverse Event Log, and further EEG studies and urine analyses for exposure to THC. The success rate has been established using a number of high-fidelity statistical methods comparing between the treatment and control groups. A detailed description of the study is provided further below (Example 3).

Apart from establishing applicability of the MCG preparations of the invention to ASD and specific partial symptoms of ASD, the presently described clinical trial (Example 3) further provides tools for establishing therapeutically effective doses and regimens of said compositions for maximization of beneficial clinical outcomes and avoidance of side effects and cross-drug interaction. In other words, this study provides an exemplary framework for applying the compositions of the invention in a rationalized manner to achieve more effective and safe treatment of ASD.

In terms of therapeutic methods, the compositions of the invention can be administered via oral or topical routes, such methods are particularly advantageous for pediatric patients. Sublingual administration the compositions of the invention has been presently demonstrated. Another advantageous form of these compositions is in the form of a transdermal patch, administered apart or together with the oral dosage forms.

As has been presently exemplified, the compositions and methods of the invention can be applied as combination therapies with other drugs, In ASD the predominant drugs are used off label with original indications for other seemingly related conditions, such as attention deficit hyperactivity disorder (ADHD), sleep disturbances or depression. Notable examples include: selective serotonin re-uptake inhibitors (SSRIs) including Fluoxetine and Sertraline; antipsychotic drugs such as Risperidone, Cozapine and Aripiprazole for treating autism-related irritability; D2 receptor antagonists such as Haloperidol for improving cognition; Oxytocin for improving social behavior; and more recently secretin, methylphenidate; and other drugs.

It is conceived that the compositions and methods of the invention, for being efficient and relatively safe, when applied as combination therapies can lead to a reduced intake of concurrent drugs, and thereby mitigate or reduce their adverse effects.

Ultimately, it is conceived that the presently proposed compositions and methods could be successfully applied as monotherapies for the treatment of ASD, or specific ASD symptoms.

### DESCRIPTION OF SPECIFIC EMBODIMENTS

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

Tetrahydrocannabinol (THC) refers herein to a class of psychoactive cannabinoids characterized by high affinity to CB1 and CB2 receptors, having a molecular formula C₂₁H₃₀O₂, an average mass of approximately 314.46 Da, and a general structure of Formula I.

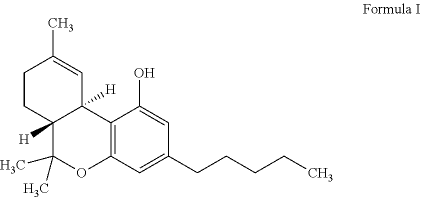 [SMILES: CCCCCc1cc(O)c2c(c1)OC(C)(C)[C@@H]1CCC(C)=C[C@@H]21]

Cannabidiol (CBD) refers herein to a class of non-psychoactive cannabinoids with a low affinity to CB1 and CB2 receptors, having a formula C₂₁H₃₀O₂, an average mass of approximately 314.46 Da, and a general structure of Formula II.

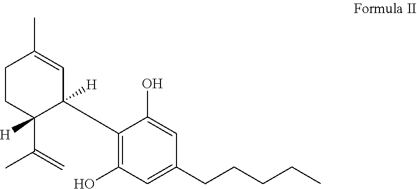 [SMILES: C=C(C)[C@@H]1CCC(C)=C[C@H]1c1c(O)cc(CCCCC)cc1O]

The terms THC and CBD herein further refer to isomers, derivatives, or precursors of these molecules, such as (−)-trans-Δ9-tetrahydrocannabinol (Δ9-THC), Δ8-THC, and Δ9-CBD, and further to THC and CBD derived from their respective 2-carboxylic acids (2-COOH), THC-A and CBD-A.

In the broadest sense, the terms THC and CBD can refer to a synthetic or semi-synthetic or a natural cannabinoid (i.e. purified or extracted from a cannabis plant). THC responsible for the psychoactive (‘high’) effect of cannabis and is known to relieve pain. CBD has no psychoactive effects and moderates the euphoric effect of THC, it is known to reduce inflammation and nausea.

Cannabis-based and cannabis-derived, these terms herein are interchangeable and signify a composition or a constituent thereof purified or extracted from a cannabis plant using known technologies known in the art. These terms can further relate to a crude dry plant material. Regarding extracts, there are number of methods for producing a concentrated cannabis-derived material, e.g., a filtration, an ice water extraction, butane extraction or CO₂ extraction processes, and oil extracts made by solvent evaporation. Certain oil extracts from the cannabis strain Avidekel are presently exemplified.

Therapeutic agent denotes herein a broad range of agents from various groups, predominantly used off-label for specific symptoms of ASD, for example: selective serotonin re-uptake inhibitors (SSRIs) such as Fluoxetine and Sertraline; antipsychotic drug such as Risperidone, Clozapine and Aripiprazole for treating autism-related irritability; D2 receptor antagonists such as Haloperidol for cognition; Oxytocin for social functioning; and more recently Secretin, Methylphenidate and other drugs.

The terms therapeutic dose or therapeutically effective dose, which herein are interchangeable, relate to doses of the presently described compositions, in any dosage form, capable of producing an improvement of at least one symptom of ASD according to clinically accepted criteria using tests and questionnaires as shown in Examples 2-3. Such improvement can be further evaluated according to severity scales. Thus an improvement in this context relates to a type and/or a number of symptoms, a severity, a frequency of symptom(s), specific groups of symptoms (partial symptoms), and/or overall manifestation of symptoms in a subject or a group evaluated by a physician or self-reporting and estimated as at least 5%, 10%, 15%, 20%, 25%, 50%, 75%, 100% or more.

Therapeutically effective amount (also pharmacologically, pharmaceutically or physiologically effective amount) herein denotes an amount of active agent needed to provide a desired level physiological response or improvement as above. The precise amount depends on numerous factors, e.g. type of an agent, activity of a composition, intended patient use (e.g. number of doses per day), patient's considerations, and others. An effective amount of an agent can be administered in one administration, or multiple administrations. The exact amount can be the result of empirical and/or individualized (case-by-case) determination.

Approximately or about, these terms herein are interchangeable, denote up to ±10% deviation from a respective measurement, or 9%, 8%, 7%, 6%, 5% or less a deviation thereof.

Thus in one of its main aspect the invention provides a cannabis-based composition for use in treating an autistic spectrum disorder (ASD) or alleviating or reducing at least one symptom of an ASD, the composition comprising CBD:THC ratio of at least about 20:1, respectively, or more in favor of CBD.

In certain embodiments the cannabis-based compositions of the invention can be used for treating, alleviating or reducing at least one partial symptom of ASD selected from self-injury, rage attacks, sleep disturbances, anxiety, mood disorders, social communication and reciprocity skills, hyperactivity, sensory sensitivities.

As stated herein, compositions of the invention comprise CBD:THC in a w/w ratio of at least about 20:1, respectively, or more in favor of CBD. In other words, compositions of the invention are highly enriched with CBD and can comprise any amount of CBD as long as the ratio is maintained (w/w).

A composition comprising CBD:THC ratio of at least about 20:1 (w/w), respectively, or more in favor of CBD, can be any such composition wherein the amount of the CBD can be high as 15-40% CBD or more (w/w), or 1-4% THC or less than 1% THC (w/w), provided that a ration of at least 20:1 (w/w) is maintained.

More specifically, compositions of the invention can comprise CBD up to at least 15%, 20%, 25%, 30%, 35%, 40%, 45%, 50% or more, or THC up to 4%, 3.5%, 3%, 2.5%, 2%, 1.5%, 1%, 0.5% or less (w/w), provided that a ratio as disclosed is maintained.

In some embodiments, compositions of the invention can comprise CBD up to at least 15%,16%,17%,18%,19%, 20%, 21%, 22%, 23%, 24%, 25%, 26%, 27%, 28%, 29%, 30%, 31%, 32%, 33%, 34%, 35%, 36%, 36%, 38%, 39%, 40%, 41%, 42%, 43%, 44%, 45%, 46%, 47%, 48%, 49%, 50% or more, and THC up to 4%, 3.5%, 3%, 2.5%, 2%, 1.5%, 1%, 0.5% or less (w/w), provided that a ratio as disclosed is maintained.

In certain embodiments the composition of the invention can comprise up to at least about 30% CBD (w/w). More specifically, they can comprise CBD up to at least 20%, 21%, 22%, 23%, 24%, 25%, 26%, 27%, 28%, 29%, 30%, 31%, 32%, 33%, 34%, 35%, 36%, 36%, 38%, 39%, 40%, 41%, 42%, 43%, 44%, 45%, 46%, 47%, 48%, 49%, 50% or more (w/w).

In some embodiments, the amount of THC is up to 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, 0.1% or less (w/w).

In certain embodiments the compositions of the invention comprise about 30% CBD and about 1.5% THC (w/w). Such compositions have been presently exemplified.

In numerous embodiments the compositions of the invention are provided in a form of an oil extract adapted for oral, topical or transdermal administrations.

In certain embodiments the compositions of the invention can be provided in oral forms for sublingual administration. Such compositions have been presently exemplified.

With respect to the oral forms, in certain embodiments the compositions of the invention can comprise at least about 24 mg CBD and 1.2 mg THC and up to at least about 240 mg CBD and 12 mg THC.

In yet other embodiments the compositions of the invention can be provided in the form of a transdermal patch or incorporated into various transdermal delivery systems by means of known in the art technologies, including first-, second- and more recent third-generation delivery systems.

The terms ‘transdermal patch’ or ‘transdermal delivery system’ (these terms are interchangeable) herein refer to a variety of systems, including first-, second- and more recent third-generation delivery systems known to be particularly advantageous for lipophilic drugs such as cannabis and cannabinoids. The compositions and dosage forms of the invention can be incorporated into these systems using of known in the art technologies.

First-generation transdermal delivery systems essentially refers to the configuration where the drug is stored in a reservoir enclosed on one side with an impermeable backing and on the other side—with an adhesive contacting to the skin. This term encompasses systems wherein the drug is dissolved in a liquid or gel-based reservoir, and also systems where the drug is incorporated into a solid polymer matrix, and also multilayer systems using impermeable, semi-permeable membranes, and systems using liquid chemical enhancers such as ethanol.

Another type of transdermal systems can employ second-generation delivery systems, i.e., those using chemical enhancers, non-cavitational ultrasound and iontophoresis.

The most recent type of transdermal delivery systems are third-generation delivery systems with targeted effects using microneedles, thermal ablation, microdermabrasion, electroporation and cavitational ultrasound.

Thus in certain embodiments the above described compositions can be derived from the cannabis strains herein designated Avidekel, as presently exemplified.

In numerous embodiments the compositions of the invention can further comprise at least one additional therapeutic agent. Relevant therapeutic agents were mentioned above.

In this form as combination therapies, in certain embodiments the compositions of the invention can alleviate or reduce at least one adverse effect or a dosing of said concurrent drug or therapeutic agent.

It is another important aspect of the invention to provide a method for treating, alleviating or reducing at least one symptom in subject suffering from an ASD, the method comprising administering to the subject a therapeutically effective amount of a cannabis-based composition comprising CBD:THC ratio of at least about 20:1, respectively.

In numerous embodiments the methods of the invention can be applied to a subject manifesting at least one partial symptom of ASD selected from self-injury, rage attacks, sleep disturbances, anxiety, mood disorders, social communication and reciprocity skills, hyperactivity, sensory sensitivities.

Efficacy of such methods can be evaluated using assessment criteria, such as: CGI-I questionnaire, SP-2 questionnaire CSHQ questionnaire, ABC-C questionnaire, as presently exemplified, and other methods

As stated herein, the methods of the invention apply cannabis-based compositions comprising CBD:THC in a w/w ratio of at least about 20:1, respectively, or more in favor of CBD, or in other words compositions highly enriched with CBD.

The compositions of the invention have been described in detail above. Thus the methods using the compositions of the invention can apply compositions comprising CBD:THC ratio of at least about 20:1 (w/w), respectively, or more in favor of CBD, or any such composition wherein the amount of the CBD can be high as 15-40% CBD or more (w/w), or 1-4% THC or less than 1% THC (w/w), provided that a ration of at least 20:1 (w/w) is maintained.

The methods of the invention can apply compositions comprising CBD up to at least 15%, 20%, 25%, 30%, 35%, 40%, 45%, 50% or more, or THC up to 4%, 3.5%, 3%, 2.5%, 2%, 1.5%, 1%, 0.5% or less (w/w), provided that a ratio as disclosed is maintained.

In some embodiments, the methods can apply compositions comprising CBD up to at least 15%, 16%, 17%, 18%, 19%, 20%, 21%, 22%, 23%, 24%, 25%, 26%, 27%, 28%, 29%, 30%, 31%, 32%, 33%, 34%, 35%, 36%, 36%, 38%, 39%, 40%, 41%, 42%, 43%, 44%, 45%, 46%, 47%, 48%, 49%, 50% or more, and THC up to 4%, 3.5%, 3%, 2.5%, 2%, 1.5%, 1%, 0.5% or less (w/w), provided that a ratio as disclosed is maintained.

In certain embodiments the methods of the invention can apply compositions comprising up to at least about 30% CBD (w/w). More specifically, they can apply compositions comprising CBD up to at least 20%, 21%, 22%, 23%, 24%, 25%, 26%, 27%, 28%, 29%, 30%, 31%, 32%, 33%, 34%, 35%, 36%, 36%, 38%, 39%, 40%, 41%, 42%, 43%, 44%, 45%, 46%, 47%, 48%, 49%, 50% or more (w/w).

In some embodiments, the amount of THC is up to 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, 0.1% or less (w/w).

In certain embodiments the methods of the invention can apply compositions comprising about 30% CBD and about 1.5% THC (w/w). Applicability of such methods has been presently exemplified.

In numerous embodiments the methods of the invention apply cannabis-based compositions in the form of oil extracts.

In certain embodiments all the above methods can apply cannabis-based compositions derived from the cannabis strain herein designated Avidekel, as exemplified here.

In numerous embodiments the methods of the invention apply the cannabis-based compositions administered via oral, topical or transdermal routes.

In certain embodiments the methods of the invention use sublingual administrations of the cannabis-based compositions.

In yet further embodiments, the methods of the invention use cannabis-based compositions administered sublingually in therapeutically effective doses in the range from at least about 20 mg CBD and 1 mg THC and up to at least about 240 mg CBD and 12 mg THC per administration.

More specifically, in terms of the two main cannabinoids, CBD and THC, the therapeutically effective dose can be in the range between about 20, 40, 60, 80, 100, 120, 140, 160, 180, 200, 220, 240, 260, 280, 300 mg CBD and 1, 2, 3, 4, 5, 6, 7, 8, 9, 10, 11, 12, 13, 14, 15 mg THC per administration.

In numerous embodiments the methods of the invention apply the cannabis-based compositions in a regimen of at least 1-2 times a day, or more, in the morning and/or evening, the morning or evening doses being equal or distinct.

The oral dosage forms of the invention are particularly applicable for establishing a personalized therapeutically effective daily amount of CBD/THC by means of a titration, for example by daily administering of 1-2 drops of the oral dosage form of the invention and increasing the dose every 3 days in the course of at least 1 week or more. (see Table 1).

In this connection the oral dosage forms of the invention are particularly useful not only in terms of efficacy but also in terms of safety to avoid manifestation of potential side effects of cannabis. Side effects of cannabis are not numerous and most are mild. Cannabis consumption can be accompanied by a feeling of euphoria and may cause two types of side effects, physiological and cognitive.

Physiological effects can include dizziness, irregular heartbeat (faster or slower), weakness, lower blood pressure and blood sugar levels, increased appetite, red eyes, tiredness, lack of coordination, lack of balance and dryness in mucous membranes such as the mouth and eyes. Cognitive side effects relate to short-term memory impairment; lose a train of thought and distortions in the perception of time and space. Regular use of large amounts can lead to cognitive impairment, particularly in in youth. Side effects usually dissipate shortly after a reduction of therapeutic dose.

Side effects occurring from overdose usually require special attention. These may include: fainting, significant changes in blood pressure, in pulse, in blood sugar levels, or in respiration rates. A high dose cannabis can cause in some cases, especially for people who are pre-disposed, a temporary psychotic attack, anxiety, delusions, or hallucinations.

In numerous embodiments the methods of the invention are applied as combination therapies further comprising at least one additional therapeutic agent administered simultaneously or in succession with the cannabis based-compositions. Applicability of such methods has been presently exemplified.

With respect to concurrent drugs or therapeutic agents, at least 4 drugs should be avoided: Astemizole, Cisapride, Pimozide and Terfenadine, due to potential cross-drug interaction and/or competition on metabolizing enzymes (CYP3A4 and CYP2C9).

In further embodiments the methods of the invention can lead to treating, alleviating or reducing at least one adverse effect or a dosing of at least one concurrent drug or therapeutic agent. Relevant agents were referred to above.

In certain embodiments the methods of the invention can be applied as monotherapies for ASD, including alleviation or reduction of specific or partial ASD symptoms.

In yet another aspect the invention provides use of a cannabis-derived material for manufacture of a medicament for treating ASD or alleviating or reducing at least one symptom in a subject suffering from an ASD, said material comprises CBD:THC ratio of at least about 20:1, respectively.

In numerous embodiments the medicament as above can be further indicated for alleviation or reduction of at least one partial symptom of ASD selected from self-injury, rage attacks, sleep disturbances, anxiety, mood disorders, social communication and reciprocity skills, hyperactivity, sensory sensitivities.

The following examples are put forth so as to provide those of ordinary skill in the art with a complete disclosure and description of how to make and use the methods and compositions of the invention, and are not intended to limit the scope of what the inventors regard as their invention.

### EXAMPLES

### Abbreviation Term

### AE Adverse Events

### ASD Autism Spectrum Disorder

### CBD Cannabidiol

### IP Investigational Product

### MGC Medical Grade Cannabis

### SAE Serious Adverse Events

### Example 1

Investigational Product (IP)

1.1 Characterization of Medical Grade Cannabis (MGC)

The investigational product (IP) in this protocol is Medical Grade Cannabis (MGC) made from plant material from the strain Avidekel administrated as liquid extract in a form of oil, with the main active components: 30% CBD and 1.5% Δ9-THC.

In general, preparation methods of a cannabis oil involve an extraction step, often followed by a solvent evaporation step, meant to make cannabinoids and other beneficial components such as terpenes available in higher concentrations. Cannabis oil is consumed orally with a controlled number of drops applied under the tongue several times a day.

More specifically, an extract of Avidekel flowers is dissolved in olive oil. It terms of the two main cannabinoids, Avidekel oil usually comprises Δ9-THC and CBD in a ratio of about 1:20 and at concentrations of about 30% CBD and about 1.5% Δ9-THC. Each Avidekel oil drop is about 0.04 ml in volume containing about 12 mg CBD and 0.6 mg Δ9-THC.

More broadly characterized, Avidekel oil contains: 45% olive oil, 30% CBD, 1.5% THC, 0.5% CBG, <0.1% CBN, <1.5% CBC, <0.5% CBDV, with unidentified cannabinoids reaching up to 21%, and also certain percentages of terpenes, flavonoids, waxes and chlorophyll.

The IP is further subjected to fine tuning of CBD/THC concentrations using pure CBD and/or olive oil (purchased from the company “Zeita”) are added to reach the necessary ratio (1:20) and the required concentration of CBD (30%). In the following studies the IP was administered to ASD patients as add-on to the standard of care medications.

The IP can be stored at room temperature.

1.2 Administration

The treatment includes sublingual administrations of the IP in the form of drops twice a day.

1.3 Dosing and Titration Period

In order to reach the optimal dose for each patient, a titration period is required, as the number of oil drops and the timing can vary between subjects. Titration period of each patient varies and can last for up to 4 weeks, and during this time the impact of treatment on the disease is not as optimal as when the dose is balanced between maximum impact on the symptoms and minimal side effects.

The initial dose is one drop of oil under the tongue twice a day (morning and evening) for three days and then two drops, twice a day, for three more days, etc. The dose is gradually increased depending on the effect and tolerability of each patient. Morning and evening doses can have different effects. The subjects continue titration until an adverse reaction is experienced, or until a maximum dosage is reached, e.g., 10 drops per administration (12 mg of Δ9-THC and 240 mg of CBD). If an adverse reaction occurs, the patient is tapered down one level to a pre-adverse reaction dose. See Table 1 summarizing titration schedule.

TABLE 1
Titration schedule demonstrating titration of one administration to maximal dose

| | Sunday | | Monday | | Tuesday | | Wednesday | | Thursday | | Friday | | Saturday | |
|---|---|---|---|---|---|---|---|---|---|---|---|---|---|---|
| Week-1 | 1.2 | mg THC | 1.2 | mg THC | 1.2 | mg THC | 2.4 | mg THC | 2.4 | mg THC | 2.4 | mg THC | 3.6 | mg THC |
| | 24 | mg CBD | 24 | mg CBD | 24 | mg CBD | 48 | mg CBD | 48 | mg CBD | 48 | mg CBD | 72 | mg CBD |
| Week-2 | 3.6 | mg THC | 3.6 | mg THC | 4.8 | mg THC | 4.8 | mg THC | 4.8 | mg THC | 6 | mg THC | 6 | mg THC |
| | 72 | mg CBD | 72 | mg CBD | 96 | mg CBD | 96 | mg CBD | 96 | mg CBD | 120 | mg CBD | 120 | mg CBD |
| Week-3 | 6 | mg THC | 7.2 | mg THC | 7.2 | mg THC | 7.2 | mg THC | 8.4 | mg THC | 8.4 | mg THC | 8.4 | mg THC |
| | 120 | mg CBD | 144 | mg CBD | 144 | mg CBD | 144 | mg CBD | 168 | mg CBD | 168 | mg CBD | 168 | mg CBD |
| Week-4 | 9.6 | mg THC | 9.6 | mg THC | 9.6 | mg THC | 10.8 | mg THC | 10.8 | mg THC | 10.8 | mg THC | 12 | mg THC |
| | 192 | mg CBD | 192 | mg CBD | 192 | mg CBD | 216 | mg CBD | 216 | mg CBD | 216 | mg CBD | 240 | mg CBD |

Titration is stopped if any of the following occurs:
fainting or lightheadedness as indicated by falling, worsening gait instability, or marked decline in motor function
noticed changes in blood pressure
noticed changes in pulse
noticed changes in blood sugar levels
noticed changes in respiration rates
noticed behavioral changes for example aggressiveness
new neurologic complaints or findings
physician's decision.

1.4 Concomitant Medications

Cannabis and cannabis preparations are considered relatively safe and non-addictive, with only minor passing effects in large doses. Additional attention should be given to potential allergic reactions. The IP can be added to the patient's current treatment regime. All changes in concurrent drugs consumption should be monitored and documented.

In other words, the IP is intended as a monotherapy and a combination therapy with traditional drugs. When properly titrated and controlled for adverse reactions, i.e., under therapeutically effective dosing, the IP can reduce use of concomitant medications and their related secondary effects.

### Example 2

Retrospective Studies

2.1 Retrospective Study Using Parents Reports

2.1.1 Study Population

The study included 53 children diagnosed with ASD by DSM IV or DSM V criteria, including: 8 females (15%) and 45 males (85%) with median age of 11 (4-22) years. All patients were treated with MCG as Avidekel oil containing CBD at a concentration of 30% and 1:20 ratio of CBD:THC (see Example 1), with median daily doses of 90 (1.5-315) and 6.75 (0.5-49.5) mg for CBD and THC, respectively and for median duration of 66 (30-588) days.

2.1.2 Study Design

Biweekly follow-up telephone interviews were conducted to assess effectiveness and safety, including use of concurrent medications and any change in symptoms and possible adverse effects. Five ASD comorbidity symptoms were evaluated: a) hyperactivity symptoms b) sleep problems, c) self-injury and rage attacks, d) anxiety and mood changes, and e) information related to social communication and reciprocity skills, wherein each comorbid symptom was categorized as (1) no change, (2) an improvement or (3) a worsening as compared to the baseline during first 30 days of treatment, at 30-90 days, and after 90 days of treatment. Overall treatment effectiveness was categorized into four categories (no change, mild to moderate improvement, significant improvement or worsening) according to the parent's reports. Categorical variables were described using frequency and percentage. Continuous variables were evaluated for normal distribution using histograms and Q-Q plots.

2.1.3 Study Results

A. Self-Injury and Rage Attacks

Available data on N=34 patients at the last follow up interview indicated: an improvement in 67.6%, no change in 23.5%, and worsening of symptoms in 8.8%.

B. Sleep Problems

Available data on N=21 patients indicated: an improvement in 71.4%, no change in 23.8, and worsening of symptoms in 4.7% (1 patient).

C. Anxiety and Mood Changes

Available data on N=17 patients indicated: an improvement in 47.1%, no change in (29.4%), and worsening in 23.5%.

D. Social Communication and Reciprocity Skills

Available data on N=15 patients indicated: an improvement with regard to at least one skill in 86.7%, no change in 13.3%, worsening was not reported.

E. Hyperactivity

Available data on N=30 indicated: an improvement in 70%, no change in 26.6%, and worsening in 3.3%.

F. Overall Change

Overall change in ASD (or comorbidities) symptoms was examined in 51 patients. Mild to moderate improvement was reported in 31.4%, significant improvement—in 43.1%, no change—in 21.6% and worsening—in 3.9%. Two patients did not provide adequate reports.

F. Adverse Events

Most frequent adverse effects were somnolence (n=7), decrease in appetite (n=6), and drowsiness (n=5). All adverse effects were transient and resolved spontaneously. Five families discontinued follow-up at different time points. Two families reported ineffectiveness and chose to stop treatment.

2.1.4 Conclusions

Avidekel oil as in Example 1 is effective in improving partial and overall symptoms of ASD. Its effects were further evaluated in a controlled randomized clinical study.

2.2 Retrospective Study Using Clinical Records

2.2.1 Study Population

The study included 190 ASD patients treated with the MCG of the invention (IP) applied under the tongue, out of which 123 patients received treatment for six months. The average age was 12.8*6.9 years (82% under the age of 18), with the distribution of 82% boys and 18% girls, and reported comorbidity of epilepsy in 14.7% of the patients. At the baseline, the main symptoms requiring therapy were: restlessness (90.5%), rage attacks (79.5%) and nervousness (78.9%).

2.2.2 Study Results

Of 67 patients treated with MCG for at least 6 months, 40 (60%) reported an improvement; 35%—a significant improvement, 45%—a moderate improvement, and less than 1%—a slight improvement, about 12% reported no change in their condition, and one patients reported a slight deterioration. Eighteen patients (45%) reported certain side effects; the most common was sleepiness (10%). The main findings are summarized in Tables 2-4.

TABLE 2
Response in specific symptoms or disabilities in ASD after 6 months treatment with IP

| | Sleep N (%) | | | Ability to dress and shower N(%) | | | Ability to concentrate N (%) | | |
|---|---|---|---|---|---|---|---|---|---|
| | Before | After | p value | Before | After | p value | Before | After | p value |
| Severe | 44 (47.3) | 2 (2.2) | <0.001 | 33 (35.5) | 23 (24.7) | <0.01 | 75 (80.6) | 21 (22.6) | <0.001 |
| difficulty | | | | | | | | | |
| Moderate | 18 (19.4) | 27 (29.0) | | 17 (18.3) | 17 (18.3) | | 11 (11.8) | 41 (44.1) | |
| difficulty | | | | | | | | | |
| No | 28 (30.1) | 39 (41.9) | | 24 (25.8) | 39 (41.9) | | 2 (2.2) | 11 (11.8) | |
| difficulty | | | | | | | | | |
| Good | 2 (2.2) | 15 (16.1) | | | | | 0 | 10 (10.8) | |
| Very Good | 1 (1.1) | 8 (8.6) | | | | | 0 | 3 (3.2) | |

TABLE 3
Response in certain partial symptoms of ASD after 6 months treatment with IP

| | Total N (%) | | Symptom | | Improvement | | No change or | |
|---|---|---|---|---|---|---|---|---|
| Restlessness | 170 | (90.4) | 1 | (1.2) | 71 | (89.8) | 7 | (8.8) |
| Rage attacks | 150 | (79.8) | 1 | (1.3) | 65 | (89.0) | 7 | (9.5) |
| Agitation | 148 | (78.7) | 1 | (1.4) | 57 | (83.8) | 10 | (14.7) |
| Sleep problems | 113 | (60.1) | 9 | (19.5) | 27 | (58.6) | 10 | (21.7) |
| Speech impairment | 113 | (60.1) | — | | 15 | (30) | 35 | (70) |
| Cognitive impairment, | 91 | (48.4) | — | | 15 | (27.2) | 40 | (72.7) |
| Anxiety, | 69 | (36.7) | — | | 24 | (88.8) | 3 | (11.1) |
| Incontinence | 51 | (27.1) | 2 | (9.0) | 7 | (31.8) | 13 | (59.0) |
| Seizures | 23 | (12.2) | 2 | (15.3) | 11 | (84.6) | — | |
| Limited mobility | 17 | (9.0) | 2 | (18.1) | — | | 9 | (81.8) |
| Constipation | 15 | (8.0) | 1 | (12.5) | 6 | (62.5) | 2 | (25) |
| Tics | 15 | (8.0) | 1 | (20.0) | 4 | (80.0) | — | |
| Digestion problems | 14 | (7.4) | 1 | (12.5) | 5 | (62.5) | 2 | (25.0) |
| Increased appetite | 14 | (7.4) | 1 | (33.3) | 1 | (33.3) | 1 | (33.3) |
| Lack of appetite | 14 | (7.4) | 2 | (40.0) | 1 | (20.0) | 2 | (40.0) |

| | | | | | | |
|---|---|---|---|---|---|---|
| Depression | 10 | (5.3) | — | 5 | (100.0) | — |

TABLE 4
Response in intake of concurrent medications after 6 months treatment with IP

| | | Stopped | | Dosage | No | | Dosage | New |
|---|---|---|---|---|---|---|---|---|
| Medication family | Total | medication | | decreased | change | | increased | medication |
| Antipsychotics, n (%) | 55 | 11 | (20) | 3 (5) | 41 | (75) | 0 | 0 |
| Antiepileptics, n (%) | 46 | 6 | (13) | 0 | 35 | (76) | 2 (4.5) | 3 (6.5) |
| Antidepressants, n (%) | 10 | 3 | (30) | 0 | 4 | (40) | 1 (10) | 2 (20) |
| Hypnotics and sedatives, n (%) | 10 | 2 | (20) | 1 (10) | 7 | (70) | 0 | 0 |
| Anxiolytics, n (%) | 7 | 2 | (28) | 0 | 5 | (72) | 0 | 0 |
| Anticholinergic agents, n (%) | 5 | 1 | (20) | 0 | 4 | (80) | 0 | 0 |
| Beta blocking agents, n (%) | 3 | 2 | (66.6) | 0 | 1 | (33.3) | 0 | 0 |
| Laxatives, n (%) | 3 | 2 | (66.6) | 0 | 1 | (33.3) | 0 | 0 |
| Psychostimulants, agents used for ADHD | 3 | 1 | (33.3) | 0 | 1 | (33.3) | 0 | 1 (33.3) |

| | | | | | | | |
|---|---|---|---|---|---|---|---|
| Drugs for peptic ulcer and | 2 | 0 | 0 | 2 | (100) | 0 | 0 |
| Opioids, n (%) | 2 | 0 | 0 | 2 | (100) | 0 | 0 |
| Other analgesics and antipyretics, n (%) | 2 | 0 | 0 | 2 | (100) | 0 | 0 |

Conclusions: Avidekel oil is effective in improving partial and overall symptoms of ASD, with relatively minimal side effects and high compliance, suggesting tolerability. A significant number of parent reported on an improved quality of life.

### Example 3

A Double-Blind, Placebo Controlled Randomized Trial

3.1 Primary Objective

To assess the efficacy and safety of MGC oil vs. placebo in relieving symptoms associated with ASD.

3.2 Secondary Objectives
i. To evaluate the safety and tolerability of cannabis in children diagnosed with ASD
ii. To assess the efficacy of MGC in reducing disruptive behaviors, sleep problems, and sensory sensitivities among children diagnosed with ASD, and in improving their quality of life;
iii. To assess the effects of MCG on brain activity depicted by a sleep EEG exam.

3.3 Working Hypothesis

Administration of MGC to children diagnosed with ASD will lead to a significant improvement of symptoms associated with ASD in comparison with placebo, wherein placebo oil contains olive oil and chlorophyll instead of Avidekel extract.

3.4 Primary Endpoints

3.4.1 Primary Efficacy Endpoint

Superiority in reducing symptoms of ASD as described and quantified via parent questionnaires and clinical assessment held by a specialized physician, in five main categories—sensory behavior, social relating, body and object use, language and communication skills, social and adaptive skills. Clinical estimation is held via the Aberrant Behavior Checklist-Community (ABC-C) questionnaire, performed at the beginning and end of each phase.

*In this and subsequent evaluations comparisons are held between clinical estimations after treatment with MCG versus placebo for both arms.

3.4.2 Primary Safety Endpoint

Tolerability and adverse effects as assessed using an adverse effect checklist evaluated in previous studies.

3.5 Secondary Endpoints

a. Superiority in reducing symptoms of ASD according to Clinical Global Impressions-Improvement (CGI-I), estimated by a physician at the beginning and end of each phase.

b. Reduced symptoms of sleep disturbances according to Children's Sleep Habit Questionnaire (CSHQ), estimated by parents at the beginning and end of each phase.

c. Reduced symptoms of sensory sensitivities according to Sensory Profile II (SP-2) questionnaire, estimated by parents at the beginning and the end of each phase, while comparing within each arm according to baseline and between both arms.

Changes in the amount of time that children look at faces in movies with social information, i.e., an eye tracking (ET) experiment, performed at the beginning and end of each phase, while comparing within each arm according to baseline and between both arms.

3.6 Trial Design

The trial is performed in two phases, including approximately 40 subjects with ASD:

Phase 1 wherein subjects are randomly assigned to receive cannabis oil (MGC oil: 30% CBD and 1.5% Δ9-THC) or placebo oil (olive oil and chlorophyll to reach identical color and texture: 0% CBD and 0% THC), and treated accordingly for 12 weeks and additional 4-week washout period.

Phase 2 wherein a crossover of trial arms takes place (patients who received cannabis oil receive placebo and vice versa)

*Clinical evaluation is performed after completing each phase.
**Patients receiving one of the following medications: Astemizole, Cisapride, Pimozide or Terfenadine, are excluded from the trial.

3.7 Trial Duration

Expected duration is 32 weeks, including:
i. 12-week treatment period of the 1^{st} phase with administration of IP (either MGC or placebo);
ii. 4-week washout without administration of IP;
iii. 12-week treatment period of the 2^{nd} phase with a crossover administration of IP (either placebo or MGC);
iv. 4-week washout period without administration IP.

3.8 Trial Population

The trial includes children between the ages of 2-8 years old with a documented diagnosis of Autism, including children with previous reports of behavioral issues characterized by aggression, anxiety, restlessness, sleep disturbances and/or self-harm as part of ASD.

The trial does not include children treated with cannabis, anti-psychotic drugs or stimulants; children with comorbidity of heart, liver, kidney or hematologic disease, children on Astemizole, Cisapride, Pimozide or Terfenadine; children suffering from epilepsy; children who underwent surgery; children with family history of psychosis and/or another mental illness.

*Patients or parent can willing withdraw from trial.

3.9 Trial Procedures

IP (MGC or placebo) are administered in two phases with washout periods, as above. IP is administered twice a day, morning and evening. Visits in the clinic are conducted at the beginning, middle, and end of each phase. See Table 5 summarizing the schedule of main clinical visits.

The following data is collected:

i. Demographic data—gender, date of birth, ethnicity

ii. Clinical data:
Medical history, patient assessments, vital signs,
Urine sample—Δ9-THC at screening and baseline
Adverse events
Concomitant medications

iii. Parent questionnaires and diaries:
Patient diary to record IP dosing (dates, times and the number of drops)
Adverse event log
Aberrant Behavior Checklist-Community (ABC-C) questionnaire
Clinical Global Impressions-Improvement (CGI-I) questionnaire
Children's Sleep Habit Questionnaire (CSHQ) questionnaire
Sensory Profile II (SP-2) questionnaire

iv. EEG sub-study (a non-mandatory arm): children are participating in three overnight EEG exams: (1) before initiating the trial (baseline), (2) after the end of phase one (week 12-16), and (3) a final exam, after the end of phase two (week 28-32).

TABLE 5
Schedule of events summarizing main clinical visits during the trial period

| | Visit 1 | | | | Visit 5 | | | |
|---|---|---|---|---|---|---|---|---|
| | Screening & | Visit 2 | Visit 3 | Visit 4 | Initiation | Visit 6 | Visit 7 | Visit 8 |
| | Enrollment | Initiation | Mid-phase 1 | End-phase 1 | of phase 2 | Mid-phase 2 | End-phase 2 | End of trial ¹ |
| Weeks | −8 weeks | of phase 1 | Week 6 | Week 12 | Week 16 | Week 22 | Week 28 | Week 32 |
| days | to 0 | 0 | 42 ± 5 | 84 ± 7 | 112 ± 10 | 154 ± 5 | 196 ± 7 | 224 ± 14 |
| Informed Consent | X | | | | | | | |
| Inclusion/Exclusion | X | X | | | | | | |
| Criteria | | | | | | | | |
| Demographics | X | | | | | | | |
| Medical history | X | | | | | | | |

Clinical Assessment

| | | | | | | | | |
|---|---|---|---|---|---|---|---|---|
| Clinical and | X | X | X | X | X | X | X | X |
| behavioral assessment | | | | | | | | |
| Vital signs ² | | X | | X | | | | X |
| Height and weight ³ | | X | | X | | | | X |
| Urine testing for | X | X | | | X | | | X |
| cannabis levels | | | | | | | | |
| Eye movement | | X | | X | X | | X | |
| examination | | | | | | | | |
| Overnight EEG | X | | | X | | | X | |
| (optional) | | | | | | | | |

Questionnaires

| | | | | | | | |
|---|---|---|---|---|---|---|---|
| ABC-C | X | X | X | X | X | X | X |
| CGI-1 | | X | X | X | X | X | X |
| CSHQ | X | | X | X | | X | |
| SP-2 | X | | X | X | | X | |

Treatment Compliance

| | | | | | | | | |
|---|---|---|---|---|---|---|---|---|
| Randomization | | X | | | | | | |
| IP dispensing | | X | | | X | | | |
| IP Return | | | | X | | | X | |
| Dispense/review | | X | X | X | X | X | X | X |
| patient dairy ⁴ | | | | | | | | |
| Concomitant | X | X | X | X | X | X | X | X |
| medication | | | | | | | | |
| Adverse events | X | X | X | X | X | X | X | X |

¹ all patients who discontinue participation before the last visit, are asked to arrive for End of Trial Visit within 7 days
² vital signs will include heart rate, blood pressure and body temperature after the patient has been sitting for 5 minutes
³ height will be measured at screening. Body weight measurement will be without shoes or heavy outwear.
⁴ Parent will document dosing and related AE

3.10 Statistical Analysis

Analyses of continuous variables use parametric methods using paired and unpaired t-test and ANOVA. Non-parametric procedures are used when parametric assumptions could not be satisfied, even after data transformation attempts, including Mann-Whitney, Wilcoxon and Spearman Correlation tests. Parametric model assumptions are assessed using Normal-plot or Shapiro-Wilks statistic for verification of normality and Levene's test for verification of homogeneity of variances. Categorical variables are tested using Pearson's χ² test for contingency tables or Fisher Exact test, as appropriate.

Continues variables with normal distribution are presented as mean and standard deviation. A 95% confidence interval is calculated when appropriate. Ordinary variables or continues variable with non-normal distribution are presented as median with an inter-quartile range. Categorical variables are presented as counts and percent of the total. Mixed models are used to account for the multiple events per patient.

3.11 Adverse Events

An Adverse Even (AE) is defined as any undesirable experience (sign, symptom, illness, abnormal laboratory value, or other medical event) occurring to a patient, that is considered related to the investigational treatment regimen prescribed as part of the clinical protocol, predefined in the clinical protocol and/or instructions for use, that is identified or worsens during a clinical trial.

A treatment related AE is defined as any adverse event, for which a causal relationship between the treatment and the event is at least a reasonable possibility, i.e., the relationship cannot be excluded. The side effects from the use of medical cannabis are not numerous and most are mild. In addition to the desired effects, cannabis consumption can be accompanied by a feeling of euphoria and may cause two types of side effects, physiological and cognitive:
Physiological effects may include dizziness, irregular heartbeat (faster or slower), weakness, lower blood pressure and blood sugar levels, increased appetite, red eyes, tiredness, lack of coordination, and dryness in mucous membranes as the mouth and eyes.
Cognitive side effects may include short-term memory impairment such as loss of train of thought and distortions in the perception of time and space. Regular use of large amounts can lead to cognitive impairment, but this effect dissipates when use is ceased. The side effects usually dissipate shortly after the patient becomes accustomed to the product.

Side effects usually occurring from overdose that require special attention: fainting, significant changes in blood pressure, in pulse, in blood sugar levels, or in respiration rates. A high dose of the product can in some cases, for people that are pre-disposed, cause a temporary psychotic attack, anxiety or delusions.

A serious unanticipated AE is one when the patient outcome is one of the following: Death, Life-threatening, Hospitalization (initial or prolonged), Disability—significant, persistent, or permanent change, impairment, damage or disruption in the patient's body function/structure, physical activities or quality of life, Congenital anomaly or requires intervention to prevent permanent impairment or damage.

The following categories of intensity of unanticipated AE are used:
Mild: awareness of a sign or symptom that does not interfere with the patient's usual activity or is transient, resolved without treatment and with no sequelae;
Moderate: interferes with the patient's usual activity, but the patient is still able to function;
Severe: events that interrupt a patient's usual daily activity and generally require a systemic device therapy or other treatment.
   * All AE are reported throughout the entire time that the patient remains active in the trial.

Protocol synopsis is provided in Table 6 below.

TABLE 6
Protocol synopsis

| DESIGN | A Double blinded, placebo controlled, randomized crossover trial. | |
|---|---|---|
| INVESTIGATIONAL | MCG oil is supplied as Avidekel oil with a concentration of 30% CBD and | |
| PRODUCT (IP) | 1.5% Δ⁹-Tetra-Hydrocannabinol (Δ9-THC); as opposed to placebo oil | |
| | without cannabinoids. The volume of every drop of the cannabis oil is 0.04 | |
| | ml containing 12 mg CBD and 0.6 mg Δ9-THC. Patients receive drops of | |
| | cannabis oil according to the trial procedures. IP is given in addition to | |
| | standard of care treatment. | |
| PHASE | IIa | |
| OBJECTIVES | To assess the efficacy and safety of MCG oil, vs. placebo, in relieving | |
| | symptoms in children diagnosed with Autism Spectrum Disorder. | |
| POPULATION | Approximately 40 children between the ages of two to eight years old with | |
| | a documented diagnosis of Autism. The trial population is randomized. | |
| PRIMARY | Superiority in reducing symptoms of ASD as described and quantified via | |
| ENDPOINT | parent questionnaires and clinical assessment held by a specialized | |
| | physician, in five main categories - sensory behavior, social relating, body | |
| | and object use, language and communication skills, and social and adaptive | |
| | skills. Clinical estimation is held via the Aberrant Behavior Checklist- | |
| | Community (ABC-C) questionnaire. Clinical estimation is performed at the | |
| | beginning and the end of each phase, while comparing results after | |
| | treatment with MCG versus after treatment with placebo for both arms. | |
| SECONDARY | Superiority in reducing symptoms of ASD as described and | |
| ENDPOINTS | quantified via parent questionnaire - The Clinical Global | |
| | Impressions-Improvement (CGI-I). Clinical estimation are | |
| | performed at the beginning and the end of each phase while | |
| | comparing results after treatment with MCG versus after | |
| | treatment with placebo for both arms. | |
| | Reduced symptoms of sensory sensitivities assessed by the | |
| | Sensory Profile II (SP-2) questionnaire. Parents answer the | |
| | questionnaire at the beginning and the end of each trial phase and | |
| | comparisons are held within each arm according to baseline and | |
| | between both arms. | |
| | Changes in eye movements and tracking as participants observe | |
| | movies with social information. An eye tracking (ET) experiment | |
| | is performed at the beginning and end of each phase to examine | |
| | changes in the way the children observe movies with social | |
| | information (e.g. children playing). Comparisons are made within | |
| | each arm according to baseline and between both arms. | |
| | Changes in the sleep architecture (e.g., amount of REM sleep) and | |
| | brain activity during sleep of participating children are assessed | |
| | by an overnight EEG exam. Comparison are made within each | |
| | arm according to baseline and between both arms. | |
| INCLUSION | Children ages two to eight years old with a documented diagnosis | |
| CRITERIA | of ASD. | |
| | Children with a previous report of behavioral issues characterized | |
| | by aggression, anxiety, restlessness, sleep disturbances and/or | |
| | self-harm, all as a part of the ASD, as documented in previous | |
| | clinical estimation and examination. | |
| EXCLUSION | Children treated with cannabis, anti-psychotic drugs or stimulants. | |
| CRITERIA | Children with a comorbidity of heart, liver, kidney or hematologic | |
| | disease. | |
| | Children treated with one of the following drugs: Astemizole, | |
| | Cisapride, Pimozide or Terfenadine. | |
| | Children suffering from epilepsy | |
| | Family history of psychosis and/or another mental illness. | |
| | Physician's professional opinion. | |
| | Children who underwent surgery during the 30 days prior to the trial. | |
| | Children that are participating in another trial which includes any | |
| | intervention. | |
| PROCEDURES | Visit 1 - Screening and Enrollment | |
| | Medical history is assessed, patients who do not have a general and | |
| | neurologic exam during the past year previous to the initiation of the trial | |
| | are referred to their physician. Clinical and behavioral assessment are | |
| | performed by a physician, vital signs and weight and height are measured, | |
| | and a urine sample are taken. All concomitant medications are documented. | |
| | Visit 2 - Randomization and Initiation of Phase 1 | |
| | Clinical and behavioral evaluation is held by a physician, including the | |
| | following questionnaires: | |
| | The Aberrant Behavior Checklist-Community (ABC-C) | |
| | The Children's Sleep Habit Questionnaire (CSHQ) | |
| | Sensory Profile II (SP-2) Questionnaire | |
| | A urine sample is taken for the presence of cannabis. The patients | |
| | participate in a 10-minute eye tracking test. The IP (drug or placebo based | |
| | upon randomization) is administered by the parents with the first dose | |
| | administration in the morning after the visit. | |
| | Visit 3 (week 6 ± 5 days) Mid Phase 1 | |
| | Clinical and behavioral evaluation is held via questionnaires (ABC-C, | |
| | CGI-1). AE and concomitant medications are reported and documented. | |
| | Visit 4 (week 12 ± 7 days) End of Phase 1 | |
| | Clinical and behavioral evaluation will be held via questionnaires (ABC-C, | |
| | CGI-1, CSHQ and SP-2), vital signs and weight are measured, including | |
| | reporting on AE and concomitant medications. Patients participate in a 10- | |
| | minute eye tracking test. Immediately after the visit, a four-week washout | |
| | period begins. | |
| | Visit 5 (week 16 ± 10) Crossover and Initiation of Phase 2 | |
| | IP is crossed over, those who received placebo now receive MCG and vice | |
| | versa. Clinical and behavioral evaluation is held via questionnaires | |
| | (ABC-C, CGI-1, CSHQ and SP-2), urine sample is taken for the presence | |
| | of cannabis. Patients participates in a 10-minute eye tracking test. Further | |
| | data include documentation of AE and concomitant medications. | |
| | Visit 6 (week 22 ± 5) Mid-Phase 2 | |
| | Clinical and behavioral evaluation is held via questionnaires (ABC-C, | |
| | CGI-1) and further reporting on AE and concomitant medications. | |
| | Visit 7 (week 28 ± 7) End of Phase 2 | |
| | Clinical and behavioral evaluation is held via questionnaires (ABC-C, | |
| | CGI-1, CSHQ and SP-2), vital signs and weight are measured, including | |
| | further reporting on AE and concomitant medications. Patients participate | |
| | in a 10-minute eye tracking test. | |
| | Visit 8 (week 32 ± 14) End of Trial | |
| | Clinical and behavioral evaluation is held via questionnaires (ABC-C, | |
| | CGI-1). Vital signs and weight and height are measured and a urine sample | |
| | are taken, including further reporting on AE and concomitant medications. | |
| | EEG Sub-study | |
| | This test is not mandatory, its purpose is assess the influence cannabis has | |
| | on brain activity during sleep. Three overnight EEG exams are held | |
| | throughout the trial: right after enrollment and before initiating the trial | |
| | (baseline), again right after the end of phase one (week 12-16), and a third | |
| | and final exam right after the end of phase two (week 28-32). | |
| STATISTICAL | Primary and Secondary Endpoint Analysis | |
| ANALYSIS | The primary endpoint are defined as superiority in reducing symptoms of | |
| | ASD as described and quantified via parent questionnaires and clinical | |
| | assessment held by a specialized physician, in five main categories - | |
| | sensory behavior, social relating, body and object use, language and | |
| | communication skills, and social and adaptive skills. Clinical estimation is | |
| | held via the Aberrant Behavior Checklist-Community (ABC-C) | |
| | questionnaire. Clinical estimation is performed at the beginning and the | |
| | end of each phase. Comparison is held between the results after treatment | |
| | with MCG versus after treatment with placebo for both arms. | |
| | Therefore, the specific hypothesis for the primary endpoint analysis will be | |
| | as follows: | |
| | H0: p2 = p1 H1: p2 ≠ p1 | |
| | Where p1 is the rate of the primary endpoint (clinical assessment via | |
| | questionnaire) in treatment period of both trial arms, and p2 in the control | |
| | period of both trial arms. The treatment will be considered significantly | |
| | superior over control group if the H1 hypothesis is accepted at significance | |
| | level of 5% (p = 0.05) in a Z-test procedure. | |
| | The proportions of success is presented as proportions out of available | |
| | observations along with 95% Confidence Intervals. | |
| | For the secondary endpoints: | |
| | 1. | Superiority in reducing symptoms of ASD as described and |
| | | quantified via parent questionnaire - The Clinical Global |
| | | Impressions-Improvement (CGI-I) for treatment with MCG in |
| | | comparison with placebo. |
| | 2. | Reduced symptoms of sleep disturbances assessed by the |
| | | Children's Sleep Habit Questionnaire (CSHQ) for treatment with |
| | | MCG in comparison with placebo. |
| | 3. | Reduced symptoms of sensory sensitivities assessed by the |
| | | Sensory Profile II (SP-2) questionnaire for treatment with MCG |
| | | in comparison with placebo. |
| | 4. | Changes in eye movements and tracking as participants observe |
| | | movies with social information via an eye tracking (ET) |
| | | experiment for treatment with MCG in comparison with placebo. |

Similar analyses are performed with the primary outcome to compare MGC
vs. placebo in both trial arms.

1.-26. (canceled)

27. A method for treating an autistic spectrum disorder (ASD), or alleviating or reducing at least one symptom of an ASD in a subject suffering therefrom, the method comprising administering to the subject a therapeutically effective amount of a cannabis-based composition comprising Cannabidiol to Tetrahydrocannabinol ratio (CBD:THC) of at least about 20:1, respectively (w/w).

28. The method of claim 27, wherein said at least one symptom of an ASD is selected from self-injury, rage attacks, sleep disturbances, anxiety, mood disorders, social communication and reciprocity skills, hyperactivity, sensory sensitivities.

29. The method of claim 27, wherein the cannabis-based composition comprises up to at least about 30% CBD (w/w).

30. The method of claim 27, wherein the cannabis-based composition comprises about 30% CBD and about 1.5% THC (w/w).

31. The method of claim 27, wherein the cannabis-based composition is administered in the form of an oil extract.

32. The method of claim 27, wherein the cannabis-based composition is derived from a cannabis strain herein designated Avidekel.

33. The method of claim 27, wherein said administering of the cannabis-based composition is via at least one of an oral, a topical or a transdermal route.

34. The method of claim 33, wherein the oral administering consists of sublingual administering.

35. The method of claim 33, wherein the cannabis-based composition is administered in the form of a transdermal patch.

36. The method of claim 33, wherein the cannabis-based composition is administered orally or sublingually in a therapeutically effective dose in the range from at least about 20 mg CBD and 1 mg THC, and up to at least about 300 mg CBD and 15 mg THC per an administration.

37. The method of claim 27, wherein the cannabis-based composition further comprises at least one additional therapeutic agent.

38. The method of claim 27, wherein said treating an ASD, or alleviating or reducing at least one symptom of an ASD, further comprises alleviating or reducing at least one adverse effect or reducing a dosing of at least one concurrent drug in the same subject.

39. The method of claim 33, wherein the cannabis-based composition is administered orally or sublingually under a dosing regimen of at least one daily dose.

40. The method of claim 39, wherein the at least one daily dose is administered in the morning and/or in the evening, the doses being equal or distinct.

41. The method of claim 27, wherein the method is a monotherapy for an ASD.

42. The method of claim 27, wherein the method is a combination therapy for an ASD further comprising at least one additional therapeutic agent administered simultaneously or in succession with the cannabis based-composition.

43. The method of claim 42, wherein said combination therapy further comprises treating, alleviating or reducing at least one adverse effect or reducing a dosing of the at least one additional therapeutic agent.
